(19) United States
(12) Patent Application Publication
(10) Pub. No.: US 2016/0375713 A1
(43) Pub. Date: Dec. 29, 2016

(54) FINGERPRINT LIFTING SYSTEMS AND METHODS FOR BIOMETRICS AND CHEMICAL ANALYSIS

(71) Applicant: National Institute of Standards and Technology, Gaithersburg, MD (US)

(72) Inventors: Jessica L. Staymates, Germantown, MD (US); Greg Gillen, Gaithersburg, MD (US)

(21) Appl. No.: 15/263,831

(22) Filed: Sep. 13, 2016

Related U.S. Application Data
(62) Division of application No. 13/719,765, filed on Dec. 19, 2012.
(60) Provisional application No. 61/661,847, filed on Jun. 20, 2012.

(51) Int. Cl. B42D 15/00; G01N 27/62; A61B 5/1172; H01J 49/00

(52) U.S. Cl. CPC B42D 15/00; H01J 49/0036; G01N 27/622; A61B 5/1172

(57) ABSTRACT

Fingerprint lifting systems and related methods are described which enable the collected print(s) to be subjected to analytical techniques that employ relatively high temperatures. The fingerprint lifting systems include a thin layer of a heat resistant pressure sensitive adhesive.

### CROSS REFERENCES TO RELATED APPLICATIONS

This application claims priority upon U.S. provisional application Ser. No. 61/661,847 filed on Jun. 20, 2012.

### FIELD

The present disclosure relates to fingerprint analysis technology, and more particularly, to systems and methods for performing chemical analysis of fingerprints.

### BACKGROUND

The use of biometrics in general, and fingerprint recognition in particular, to identify and authenticate humans is well established. Biometrics is a group of technologies which utilize a person's unique physical or other traits as a means of confirming identity. Fingerprint capture is an important aspect of biometric technology.

In order to obtain an image of a fingerprint, typically, fingerprint powders are deposited upon a fingerprint residing on a surface. The fine particles of powder adhere to residue left from contact with the surface by friction ridge skin on a person's fingers. As will be understood, the pattern or arrangement of residue from the friction ridge skin residing on the surface, made visible by the powder, constitutes the fingerprint (or other print of interest, such as prints from palms or feet).

Fingerprint identification systems typically involve the use of a computer and associated software, which provide an identification probability for a match of a fingerprint to a previously obtained fingerprint stored in a database. In this manner, fingerprint scanning devices have been developed for capturing an image of a fingerprint.

Techniques are also known for collecting latent fingerprints left on a variety of surfaces. These techniques generally involve capturing or removing at least a portion of material constituting or representing the fingerprint of interest while concurrently maintaining the pattern of the original fingerprint.

Examples of such techniques include relatively sensitive DNA analyses of fingerprint remnants as a means to provide additional information associated with a collected fingerprint. In order to perform DNA analyses of fingerprint remnants, the collected fingerprints must be transported to a laboratory. Thus, as far as is known, field chemical analysis of collected fingerprints is presently not available.

Therefore, a need remains for techniques and associated systems in which collected fingerprints can be subjected to additional analytical methods and particularly analytical techniques which are relatively robust and vigorous, and can be performed on site.

### SUMMARY

The difficulties and drawbacks associated with previously known technology are addressed in the present systems and methods for analyzing lifted fingerprints.

In one aspect, the present subject matter provides a system for collecting (or lifting) latent fingerprints. The system comprises a substrate defining a smooth collection face. The system also comprises a layer of an adhesive disposed on the collection face of the substrate. The adhesive exhibits heat resistant properties such that upon exposure to a temperature of 200° C., the adhesive is chemically stable and does not significantly emit volatile vapors, referred to as outgassing.

In another aspect, the present subject matter provides a method of analyzing a lifted fingerprint. The method comprises providing a fingerprint lifting system including (i) a substrate defining a smooth collection face, and (ii) a layer of an adhesive disposed on the collection face of the substrate, the adhesive exhibiting heat resistant properties such that upon exposure to a temperature of 200° C., the adhesive is chemically stable and does not significantly outgas. The method also comprises contacting the layer of the adhesive of the lifting system with a fingerprint on a surface, whereby at least a portion of material constituting the fingerprint is transferred to and retained on the layer of the adhesive. The method also comprises separating the lifting system from the surface whereby the material retained on the layer of the adhesive is a lifted fingerprint. And, the method comprises subjecting the layer of the adhesive and the lifted fingerprint to an analytical operation involving exposure to a temperature of at least 200° C.

In yet another aspect, the present subject matter provides a method of analyzing a fingerprint. The method comprises obtaining a fingerprint disposed on a medium which exhibits heat resistant properties. And, the method also comprises analyzing the fingerprint disposed on the medium. The analysis involves exposing the fingerprint to a temperature of at least 200° C.

As will be realized, the subject matter described herein is capable of other and different embodiments and its several details are capable of modifications in various respects, all without departing from the claimed subject matter. Accordingly, the drawings and description are to be regarded as illustrative and not restrictive.

### BRIEF DESCRIPTION OF THE DRAWINGS

FIG. 1A is a schematic side view illustration of an embodiment of a fingerprint lifting system in accordance with the present subject matter.

FIG. 1B is a schematic perspective view of the fingerprint lifting system of FIG. 1A.

FIG. 2 is a schematic view of the fingerprint lifting system of the present subject matter in use and prior to capturing a fingerprint.

FIG. 3A is a schematic perspective view of the fingerprint lifting system and a captured fingerprint.

FIG. 3B is a schematic side view illustration of the fingerprint lifting system including a lifted fingerprint and a protective layer covering the fingerprint.

### DETAILED DESCRIPTION OF THE EMBODIMENTS

The present subject matter provides fingerprint lifting systems and associated methods, which can be subsequently used for chemical analysis, including techniques that require the sample to be heated to temperatures up to and exceeding 200° C., such as ion mobility spectrometry (IMS). IMS is a rugged and portable technique that can be used immediately in the field for example to detect contraband substances such as narcotics and explosives. Although IMS is described herein, it will be appreciated that other analytical techniques could be used such as, but not limited to, fluorescence based detection, Fourier Transform Infrared Spectroscopy (FTIR) based detection, mass spectrometry, and the like. Furthermore, it is also contemplated that the present subject matter could be utilized in conjunction with non-thermal techniques. Using the fingerprint lifting methods in accordance with the present subject matter, a latent fingerprint is developed, for example with fingerprint powders, and lifted, and then screened with IMS. The analyzed fingerprint stays intact for imaging and matching algorithms, typically performed at a later time. It will be understood that although the present subject matter is described in terms of fingerprints, the subject matter can be used in association with other regions or parts of the body which contain identifying skin ridges or patterns such as the soles of feet and palms of hands.

Currently available transparent lifting tape used to lift developed fingerprints is relatively inexpensive and readily available for crime scene technicians to use. However, such fingerprint lifting tape is not suitable for high temperature analyses such as those involving temperatures of 200° C. or greater. The typical use of fingerprint lifting tape encapsulates the analytes under the tape, precluding subsequent analysis. Also, the tape melts during the heating process and thus causes background interferences in the chemical analyzers.

In accordance with the present subject matter, a new fingerprint lifting medium eliminates these issues and provides an opportunity to chemically analyze the lifted fingerprint at a crime scene or in a laboratory. The present subject matter is useful for example, to military personnel or law enforcement in the field when they come in contact with a suspicious package that could be an improvised explosive device (IED). Such personnel could quickly brush the package for prints, lift the print with the present subject matter fingerprint lifting system, analyze the lifted prints immediately with a field-ready trace explosives detector, and save the prints for matching for subsequent determination as to the identity of people who may have handled the package.

In certain embodiments, the present subject matter systems and methods use a smooth opaque poly(tetrafluoroethylene) (PTFE) substrate coated with a transparent heat resistant and low outgassing silicone adhesive. The adhesive is sufficiently tacky to lift a powdered latent fingerprint, and the adhesive is also compatible with chemical screening techniques that use high temperatures, i.e. temperatures of 200° C. or greater, to analyze the collected material. Using a white opaque substrate is useful for contrasting with fingerprint powders, which are often black in color. One advantage of the present subject matter as compared to conventional fingerprint lifting systems and methods is that the substrate and adhesive are heat resistant and can withstand being heated to at least 200° C. and in certain embodiments, at least 250° C. without interfering with chemical analyses. Even after the heating process (which, for IMS, typically occurs for less than 10 seconds), the fingerprint stays intact and can still be useful in a fingerprint matching system.

The PTFE substrate can be cut or otherwise formed to any size necessary for the chemical analysis technique to be used, and the silicone adhesive can be applied to any specific region on the substrate. These aspects render the present subject matter useful for different instruments that use varying sample introduction methods. The adhesive can be applied using a variety of methods, including but not limited to brushing, spin-coating, and airbrushing.

A more general description of the present subject matter and its various embodiments is as follows.

### Substrate

A wide array of substrates can be used in the present subject matter fingerprint lifting systems and methods. Although PTFE is noted as a preferred material for the substrate, it will be appreciated that a wide array of other materials or combinations of materials could be used. Generally, any material which is physically and chemically stable at temperatures associated with the analyses to be performed upon the lifted prints, can be used. Thus, it is preferred that the selected substrate exhibit properties such that it is heat resistant and can withstand being heated to at least 200° C. and in certain versions, at least 250° C. without interfering with chemical analysis. Moreover, it is preferred that the selected substrate is thermally stable and does not significantly outgas or generate chemical background interferences in the contraband detectors. This is what is meant by the term “not significantly outgas” with regard to the substrate. Thus, the substrate as described herein can be heated to temperatures as high as 250° C. and remain thermally stable and not emit outgases that would interfere with the particular analysis or detector. Generally, suitable substrates include a variety of heat resistant substrates, including by not limited to NOMEX, KEVLAR, stainless steel mesh, and certain paper materials. The substrates may be either rigid or flexible.

The substrate may be opaque, transparent, or translucent. Generally, the substrate is opaque and white in color. However, the present subject matter includes substrates exhibiting different colors and/or optical characteristics.

The substrate may be provided in a wide range of shapes and sizes. Generally, the substrate is of a size suitable to accommodate one or more print(s) of interest, and yet be positioned in or used with an instrument or device that will undertake analysis of the print(s). Typically, the thickness of the substrate is from about 0.1 mm to about 1.0 mm, with about 0.3 mm to 0.4 mm being common.

### Adhesive

The adhesive used in the present subject matter exhibits heat resistant properties such that upon exposure to temperatures of up to 200° C., and in certain embodiments up to 250° C., the adhesive is chemically stable and does not significantly outgas. An indication of these characteristics is provided by the behavior of the adhesive and specifically the extent of mass loss of the adhesive and the proportion of volatile materials collected from the adhesive after heating. Specifically, upon exposure of the adhesive to temperatures of up to 125° C., the total mass loss (TML) is less than 1% and the collected volatile condensable material (CVCM) is less than 0.1% (all percentages expressed herein are based on weight percentages unless noted otherwise). This is what is meant by the term “not significantly outgas” with regard to the adhesive. Thus, the adhesives as described herein when heated to temperatures of 125° C. do not exhibit out gassing greater than these levels. These parameters are measured in accordance with ASTM E595 in which a tested sample is heated to 125° C. at reduced pressures. In certain versions of the present subject matter, the adhesive used exhibits a total mass loss (TML) upon exposure to temperatures of 125° C. of less than 0.50%, preferably less than or about 0.30% and more preferably about 0.15% or less; and a collected volatile condensable material (CVCM) of less than 0.08%, preferably less than or about 0.07%, and more preferably about 0.04% or less. In many versions of the subject matter, it is also preferred that the substrate exhibit similar or comparable properties.

In certain versions of the present subject matter, a silicone pressure sensitive adhesive is used for the adhesive layer or region. The adhesive is typically a one-part or single part pressure sensitive adhesive composition. A preferred silicone adhesive for use with the present subject matter is commercially available under the designation CV-1161 from NuSiL Technology of Carpinteria, Calif. Typical properties of that adhesive are set forth below in Table 1:

### Table 1—Typical Properties of CV-1161 Silicone Adhesive

TABLE 1
Typical Properties of CV-1161 Silicone Adhesive

| Properties | Result | ASTM |
|---|---|---|
| Uncured | | |
| Chemical Classification | MQ | — |
| Appearance | Clear Liquid | D-2090 |
| | to Translucent | |
| Non-Volatile Content | 50%-60% | D-2288 |
| Solvent | Ethyl Acetate | — |
| Cured | | |
| Total Mass Loss (TML) | 0.15%-0.30% | E-595 |
| Collected Volatile | 0.04%-0.07% | E-595 |
| Condensable Material | | |
| (CVCM) | | |

However, it will be appreciated that the present subject matter can utilize other adhesives in combination with or instead of the previously noted silicone adhesives such as CV-1161.

The adhesive can be applied or deposited upon the substrate in a variety of different techniques to produce continuous and non-continuous adhesive regions on the substrate. A continuous layer is generally preferred. A typical thickness of the adhesive layer is from about 0.01 mm to about 0.1 mm, with 0.04 mm to 0.06 mm being common. After application of the adhesive, the adhesive is cured. Curing can be performed by heating the adhesive to a temperature of about 60° C. for about 2 hours or at 200° C. for as little as 15 minutes. It has also been observed that curing can be performed by heating the adhesive to a temperature of 230° C. for 1 hour. Generally, curing at temperatures of from about 50° C. to about 250° C. will be sufficient. Different combinations of curing temperatures and times can be utilized.

Although the present subject matter is described in terms of use of an adhesive, it will be appreciated that other materials or mediums can be used.

### Protective Layer

The present subject matter also includes an optional protective layer that is disposed over or upon the adhesive layer prior to use and/or after use. The optional protective layer serves as a liner to protect or cover the adhesive layer. Thus, the protective layer prevents contamination of the adhesive layer. Immediately before use of the fingerprint lifting system, the protective layer is removed to thereby expose a face of the adhesive layer. After collecting or lifting a fingerprint, and so the print is residing on the outer face of the adhesive, a protective layer can be applied to cover the fingerprint and portions or all of the adhesive face.

A wide array of materials can be used for the protective layer. Representative examples of such materials include, but are not limited to paper, polymeric film materials, combinations of such materials, and the like. Additional nonlimiting examples of suitable materials include polyester films such as biaxially oriented polyethylene terephthalate (PET) generally known as MYLAR, and thin films of regenerated cellulose also known as CELLOPHANE. Paper such as silicone coated release paper as known in the packaging arts can also be used.

Typically, the protective layer is transparent or substantially so. For many applications, transparency is important for imaging the fingerprint for subsequent matching, but once an image of the print is captured, an opaque covering or use of a storage case is acceptable. Generally, the present subject matter includes the use of opaque or non-transparent protective layers.

### Fingerprint Powders

The present subject matter can be used with a wide array of fingerprint powders. The fingerprint powders should also be chemically stable and not significantly outgas upon exposure to conditions associated with the analytical technique(s) to be utilized.

Fingerprint powders have various formulations, and the appropriate powder should be used on the appropriate surface. For example, dark colored powders will reveal a fingerprint far better on a light surface.

Powders may be applied with a fingerprint brush, which is typically a brush with extremely fine fibers adapted to hold powder. The powder is deposited gently on the fingerprint to be revealed, without rubbing away the often delicate residue of the fingerprint itself. Powders may also be applied by blowing the powder across the fingerprint, or by pouring or otherwise dispersing the powder onto the print, and then blowing away or removing the excess.

Magnetic powders can also be used, in which a fine magnetic powder is held by a magnetic applicator, which may then be gently moved across the fingerprint. As no bristles contact the surface, this often damages the print less than other methods of developing the print.

Modern fingerprint powders have a variety of compositions, and are often a matter of personal choice by the expert using them or dictated by procedures of the relevant department or agency. Many agencies use proprietary powders produced by independent companies, and so the exact formulation of these powders is not revealed.

Some surfaces, such as organic surfaces, are not compatible with fingerprint powders and use of alternate methods is necessary. Other media, such as certain types of glue, can be “fumed” over these surfaces with fair results.

Historically, Lycopodium powder, the spores of Lycopodium and related plants, was used as a fingerprint powder.

### Additional Embodiments

The following description details additional aspects of a preferred embodiment system and method in accordance with the present subject matter.

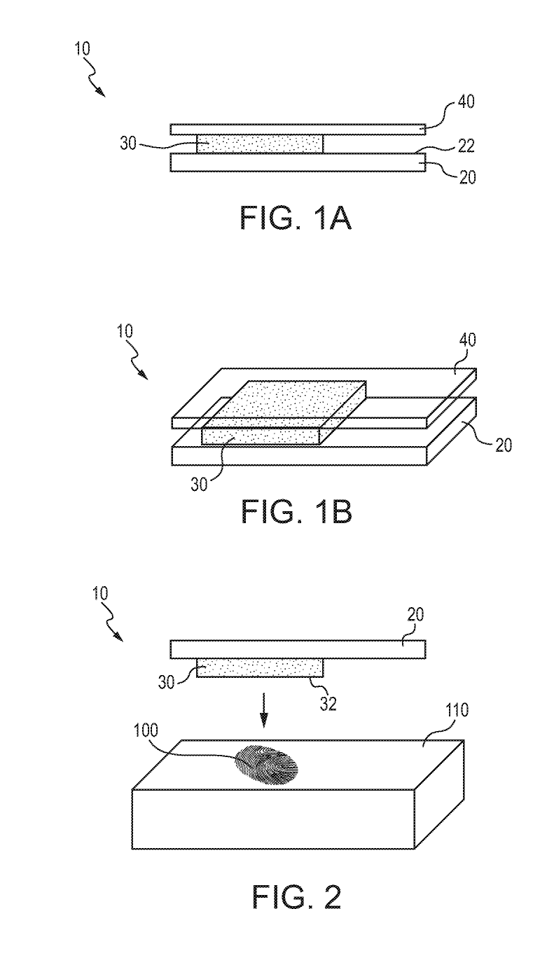

FIG. 1A is a schematic side view illustration of a fingerprint lifting system 10 in accordance with the present subject matter. FIG. 1B is a schematic perspective view of the system 10. The system 10 comprises a substrate 20 defining a smooth collection face 22, a layer of a heat resistant adhesive 30, and an optional protective layer 40. The adhesive layer 30 is disposed between the protective layer 40 and the substrate 20.

FIG. 2 illustrates the system 10 in use and prior to contact with a fingerprint 100 disposed on a substrate or surface 110. Typically, prior to use of the system 10, the fingerprint is “dusted.” This term refers to a common practice of applying or depositing a fine particulate powder, i.e. a fingerprint powder, upon the fingerprint(s) of interest to promote visibility and subsequent lifting and analysis of the print(s). The protective layer 40 is removed from the system 10 to thereby reveal an exposed face 32 of the adhesive layer 30. The system 10 is oriented such that the exposed adhesive face 32 is directed toward the fingerprint 100 of interest. The adhesive face 32 is then contacted with the fingerprint 100 such that at least a portion of the fingerprint, i.e. the oils and/or bodily materials constituting the print and generally referred to herein as the print residue, are transferred to and retained upon, the adhesive face 32. In the event that the fingerprint 100 is dusted prior to contact with the adhesive, at least a portion of the particulate material deposited on or associated with the fingerprint, is also transferred to and retained upon, the adhesive face 32.

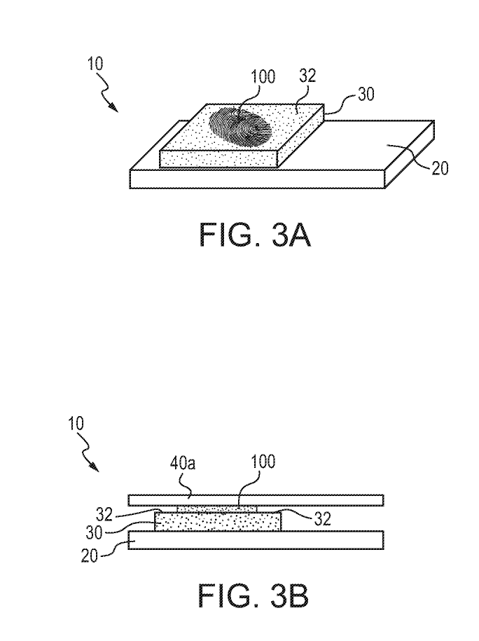

After obtaining or “lifting” of the fingerprint, the fingerprint is then retained upon the adhesive face 32 of the system 10 as shown in FIG. 3A. FIG. 3B is a schematic side view illustration of the fingerprint lifting system 10 including a lifted fingerprint 100 and a protective layer 40a positioned over and disposed on the fingerprint 100 and the adhesive face 32. It will be understood that FIG. 3B is schematic in nature and that the thickness of the fingerprint 100 is greatly exaggerated to more clearly show the arrangement of the components. Furthermore, in practice, a space or separation distance would not be maintained between the protective layer 40a and the adhesive face 32. Instead, those components would contact one another. The protective layer 40a may be the same as or different than the previously described protective layer 40.

This substrate can be immediately analyzed using a field deployable chemical analyzer. As previously noted, the analyzer may be a portable analyzer based upon IMS. After analysis, the protective layer can be carefully reattached (as shown in FIG. 3B), and the sample can be labeled accordingly and saved for scanning and matching against a known fingerprint database using a computer algorithm.

### Analytical Techniques

Use of the present subject matter system and method, enables lifted print(s) to be chemically analyzed using a wide array of techniques that may subject the lifted print(s) to temperatures as high as 200° C. and in certain instances, up to 250° C. or greater. An example of such a chemical technique is ion mobility spectrometry (IMS), which can detect trace levels of narcotics and explosives. Typically for this method, surfaces are swiped with a suitable collection material. The collection material is then inserted into the IMS sample chamber where it is heated to temperatures of 200° C. or more. After a 10 to 20 second analysis time, a display informs the user as to whether the presence of drugs or explosives was detected.

Ion mobility spectrometry is well known in the art and described for example in U.S. Pat. Nos. 7,718,960; 7,075,070; 6,797,943; and 4,551,624.

As previously noted, it will be understood that the present subject matter can be used in conjunction with a wide array of other field-deployable techniques besides IMS. The present subject matter could also be used for laboratory based techniques (not field deployable) such as a variety of mass spectrometry techniques.

### EXAMPLES

Investigations were conducted using a fingerprint capture system as described herein, to determine the feasibility of lifting a fingerprint, analyzing the lifted print for explosives, and the usefulness in a print matching system. White PTFE was cut into 1 inch (25.4 mm) by 3 inch (76.2 mm) pieces (0.015 inch (0.38 mm) thick). A heat resistant, low outgassing silicone adhesive from NuSil (CV-1161) was diluted and applied to the region of interest on the PTFE strips by airbrushing. The adhesive strips were heated to 230° C. to cure, and then were ready to use.

Latent fingerprints were placed on clean glass slides, and were brushed with fingerprint powder for development. A handful of latent prints were prepared after a volunteer handled small amounts of explosives. All prints were lifted with the fingerprint lifting substrate. The lifted prints were then scanned to create a digital image, and organized in a computer database of ‘unknown’ samples, or probes. Known fingerprints from the same unnamed volunteer were collected on five fingerprint cards using ink. These cards were scanned, and the images were cropped and organized in a gallery database of known prints. All of the images were passed through a research-grade minutiae detector and matching system. Twenty samples were compared to 60 known images, for a total of 1200 comparisons. Once a match score for all 20 samples was generated, the samples were analyzed using IMS, where each sample was heated to 230° C. for 7 seconds. The samples were then rescanned and passed through the matching system again to determine whether the heating process altered the fingerprints.

The matching outcome remained the same in over 95% of the samples. In 3% of the samples, the print changed for the better; and an initially incorrect matching result became the correct result after heating. In less than 2% of the samples, the heating process negatively affected the fingerprint matching results. Fingerprints containing explosives were screened using IMS, and explosives were detected in all 15 samples using this technique. This is significant since there are many substances being introduced into the chemical analyzer using the fingerprint lifting system including the adhesive, fingerprint powder, sebaceous materials from the print, and contamination that may be within the print. It is crucial that this combination of substances does not inhibit the response for explosives/narcotics or create a false alarm result. The present investigations demonstrated successful detection without false positive results, and successful matching using the fingerprints that had been chemically analyzed.

Although the present subject matter has been described with regards to analytical techniques employing temperatures greater than 200° C., it will be understood that the present subject matter can also be used in association with other analytical techniques such as those using temperatures less than 200° C. It is also contemplated that the present subject matter can be utilized without heating.

Many other benefits will no doubt become apparent from future application and development of this technology.

All patents, applications, standards, and articles noted herein are hereby incorporated by reference in their entirety.

As described hereinabove, the present subject matter overcomes many problems associated with previous strategies, systems and/or devices. However, it will be appreciated that various changes in the details, materials and arrangements of components, which have been herein described and illustrated in order to explain the nature of the present subject matter, may be made by those skilled in the art without departing from the principle and scope of the claimed subject matter, as expressed in the appended claims.

1-10. (canceled)

11. A method of analyzing a lifted fingerprint, the method comprising:
providing a fingerprint lifting system including (i) a substrate defining a smooth collection face, and (ii) a layer of an adhesive disposed on the collection face of the substrate, the adhesive exhibiting heat resistant properties such that upon exposure to a temperature of 200° C., the adhesive is chemically stable and does not significantly outgas;
contacting the layer of the adhesive of the lifting system with a fingerprint on a surface, whereby at least a portion of material constituting the fingerprint is transferred to and retained on the layer of the adhesive;
separating the lifting system from the surface whereby the material retained on the layer of the adhesive is a lifted fingerprint;
subjecting the layer of the adhesive and the lifted fingerprint to an analytical operation involving exposure to a temperature of at least 200° C.

12. The method of claim 11 wherein the adhesive is a silicone adhesive.

13. The method of claim 11 wherein the substrate comprises poly(tetrafluoroethylene) (PTFE).

14. The method of claim 11 wherein the adhesive exhibits a total mass loss (TML) of less than 1% as measured in accordance with ASTM E595.

15. The method of claim 11 wherein the adhesive exhibits a collected volatile condensable material (CVCM) of less than 0.1% as measured in accordance with ASTM E595.

16. The method of claim 11 wherein the analytical operation is ion mobility spectrometry (IMS).

17-21. (canceled)

22. The method of claim 11 wherein the substrate is opaque.

23. The method of claim 11 wherein the adhesive exhibits a total mass loss (TML) of less than 0.50% as measured in accordance with ASTM E595.

24. The method of claim 11 wherein the adhesive exhibits a collected volatile condensable material (CVCM) of less than 0.08% as measured in accordance with ASTM E595.

25. The method of claim 11 wherein the fingerprint lifting system further includes a protective layer on the adhesive layer.

26. The method of claim 11 wherein the substrate has a thickness of from 0.1 mm to 1.0 mm.

27. The method of claim 11 wherein the adhesive is a silicone pressure sensitive adhesive.

28. The method of claim 11 wherein the adhesive layer has a thickness of from 0.01 mm to 0.1 mm.
